(19) United States
(12) Patent Application Publication
(10) Pub. No.: US 2008/0045787 A1
(43) Pub. Date: Feb. 21, 2008

(54) MEDICAL DEVICE MADE WITH A SUPER ALLOY

(75) Inventors: James P. Snay, Republic, OH (US); Timothy E. Mackey, Marblehead, OH (US)

Correspondence Address: GANZ LAW, P.C., P O BOX 2200, HILLSBORO, OR, 97123, US

(73) Assignee: GYRUS ACMI, INC., Southborough, MA (US)

(21) Appl. No.: 11/842,811

(22) Filed: Aug. 21, 2007

Related U.S. Application Data
(60) Provisional application No. 60/839,015, filed on Aug. 21, 2006.
(60) Provisional application No. 60/823,498, filed on Aug. 24, 2006.

(51) Int. Cl. A61B 1/00

(52) U.S. Cl. 600/109

(57) ABSTRACT

A medical device with a shaft having a proximal end and a distal end, the proximal end having an operational interface, the distal end having a working element, and the shaft may include a super alloy. In one example embodiment, the shaft may include a cobalt base alloy. In another example embodiment, a medical device includes a first portion having a super alloy, and a second portion having a dissimilar metal joined to the first portion by an arc welding technique. In another possible embodiment, a first portion includes a cobalt base alloy, and a second portion includes a non-metal joined to the first portion. The inventive subject matter also includes a method for joining a cobalt base alloy to a dissimilar metal by hermetic sealing, such as provided, for example, by an arc welding technique.

### CROSS-REFERENCE TO RELATED APPLICATIONS

This application claims priority to and the benefit of U.S. provisional patent application Ser. No. 60/839,015, filed on Aug. 21, 2006, and U.S. provisional patent application Ser. No. 60/823,498, filed on Aug. 24, 2006, both by Jay Snay, et al., entitled “MEDICAL DEVICE HAVING SEMI-RIGID SHAFT,” the entire disclosure of which is hereby incorporated by reference as if set forth in its entirety for all purposes.

### BACKGROUND

The inventive subject matter disclosed herein generally relates to semi-rigid shaft or sheath constructions for medical devices that are insertable into a body cavity or passageway for performing a procedure or viewing a target site. It is particularly directed towards a medical device in the form of an endoscope. And more particularly, it is directed to a semi-rigid ureteroscope adapted to be inserted into the ureter of a patient for performing a medical examination and/or procedure. The invention may also be used in a non-medical endoscope. Hereinafter, an endoscope shaft construction primarily will be used to illustrate the inventive subject matter.

Endoscopes may be configured for a variety of applications. For example, a ureteroscope is used in the urethra, bladder, ureter or kidney. Some ureteroscopes are provided with a semi-rigid elongated shaft to enable the urologist or user to insert the distal tip through the urethra orifice and to advance the same through the bladder. Surgical procedures may be performed within the urinary system such as destroying and/or removing bladder stones, ureteral stones, kidney stones or examining calyces of the kidney. The flexibility of the tip and provision of a semi-rigid section are important features of a ureteroscope in the urology field because the anatomical path of the ureter between the bladder and the kidney is not a straight line, but often tortuous.

A problem with the conventional endoscopic systems is that they are expensive devices, with relatively short lifetimes and high-maintenance costs. Using a general-purpose endoscope for specialized recurring tasks exposes it to wear and tear, for example, during cleaning, sterilization, handling, and use. In addition, because the anatomy of the patient may be very tortuous, it is desirable to combine a number of performance features in these endoscopes, such as a relatively high level of pushability and torqueability. It may also be desirable that certain endoscopes, such as ureteroscopes, be relatively flexible. The shafts of many such endoscopes are made of stainless steel. Frequent use of these stainless steel endoscopes, with associated repeated cycles of bending and stretching, might lead to breakage of the shaft or permanent deflection. What is desired is an endoscope with a semi-rigid shaft that withstands frequent sterilization procedures, while retaining its flexibility and elasticity. Superelastic alloy materials of nickel and titanium, such as Tinel or Nitinol, for example, do not provide the rigidity required for ureteroscopes and other scopes or devices that also require flexibility. Details of typical ureteroscopes are described in U.S. Pat. No. 5,169,568 and U.S. Pat. No. 6,485,411, the disclosures of which are incorporated herein by reference. U.S. Pat. No. 5,169,568 describes a method for casting a housing around an endoscope frame. U.S. Pat. No. 6,485,411 discloses an endoscope shaft with superelastic alloy spiral frame and braid.

Some attempts have been made to address the aforementioned problems. For example, U.S. Pat. No. 5,876,330 discloses an endoscope having a shaft with two sections to provide for a combination of sufficient rigidity and flexibility. The first section of the endoscope runs along a majority of the length of the shaft and has a semi-rigid tube made of metal such as stainless steel. The second section is connected to a distal end of the first section and has a tube made of an undisclosed malleable material, allowing the tube to be pre-formed to a desired shape by hand. Another example, disclosing an elongated shaft with some ability to deflect or flex, is U.S. Pat. No. 4,986,258, which describes an endoscope utilizing a plurality of sections. The various sections may be formed by a plurality of coaxially aligned tubes of decreasing diameters or could be formed of a single elongated shaft which is machined to provide the desired tapered surface along its length with the distal end having the smaller geometrical dimensions. The joints or transitions provide zones of reduced mechanical stiffness.

The aforementioned patents describe complicated constructions made of stainless steel, and while stainless steel may be formed into a flexible structure, it is not highly fatigue resistant.

Additional problems arise when an endoscope is sterilized in an autoclave or other sterilization procedure. Such procedures help clean and sterilize endoscopes before they can be used on another patient. The procedures are performed at high temperatures and pressures that may damage and degrade the sealing systems protecting the internal parts of the endoscope. What is needed is an endoscope that stays hermetically sealed through the rigors of repeated medical autoclaving and sterilization procedures.

Some attempts have been made to provide hermetic enclosures. For example, U.S. Pat. No. 6,572,537 discloses an endoscope having a solid-state imaging pickup device with a distal tip sapphire window and a sapphire rear end cover. The cover and window are subjected to a metallization process and then joined by an airtight brazing process to metal members to form a hermetic seal. Soldered or brazed connections are used in various other places in the device to form hermetic seals. (See also U.S. Pat. Nos. 6,716,161; 6,547,722; 6,547,721; 6,425,857; 6,328,691; 6,146,326; 6,080,101; 6,030,339; 5,868,664; 5,810,713; 5,188,094; and 4,878,485.) All the foregoing patents are hereby incorporated by reference, as if set forth herein in their entireties.

Endoscope design can significantly benefit from a greater freedom for a part-by-part selection of materials, guided by the purpose of each part and the properties of the available metals and alloys. An impediment to this sought-after design freedom is that dissimilar metals or other materials may not be joinable together using known techniques, or at least not in a manner that yields a hermetically sealed joint. For example, dissimilar metals such as stainless steel, aluminum, and titanium are difficult or impossible to weld to each other via laser welding, arc welding, and similar techniques. Conventional techniques, such as laser welding, used to join dissimilar materials, such as stainless steel and cobalt base alloy, resulted in joints with micro-fractures, and were deemed not suitable or optimizable for intended autoclaving procedures.

In view of the foregoing there is a substantial need for medical devices having higher performance shafts that provide, for example, a desired balance of flexibility/rigidity, and which are fatigue resistant. There is also a substantial need for improved techniques for joining a wider range of dissimilar materials for use in medical instruments, particularly techniques that lead to hermetic joints between dissimilar materials.

### SUMMARY

The inventive subject matter offers solutions for these problems by providing improved shafts and joining techniques. Certain possible embodiments that illustrate the inventive subject matter are as follows.

In one possible embodiment, the inventive subject matter provides a semi-rigid shaft of an alloy that is semi-rigid and that is higher performance than stainless steel. In particular, the inventive subject matter discloses improved medical devices having such elongated shafts of a cobalt base alloy wherein the shaft supports operational functionalities at proximal and distal positions. The elongated shaft may be hollow or semi-hollow as in a sheath with a longitudinal channel, for example, or a solid or partially solid structure. Suitable semi-rigid alloys are cobalt base alloys. A particularly suitable cobalt base alloy is based on cobalt and chromium.

In another possible embodiment, the inventive subject matter provides a medical device that includes a shaft having a proximal end and a distal end, the proximal end having an operational interface, the distal end having a working element, and the shaft constructed using a super alloy. In the foregoing embodiment, the medical device may be configured with the following additional configuration factors: a solid shaft; a shaft including a longitudinal channel; an operational interface including a handle; a control section at the proximal end for controlling the working element; a working element including a port in communication with a channel disposed in the shaft; an imaging system disposed at the distal end of the shaft; an operational interface connected or connectable to an image processing system; an imaging system including an imaging sensor; an imaging system including an optical view port; a housing joined to the shaft at a shaft portion comprising a super alloy, and the housing portion joined to the shaft including a metal dissimilar from the super alloy; the shaft and the housing joined by an arc welding process; a Tungsten Inert Gas welding process; or a micro Tungsten Inert Gas welding process. The joining may occur via a melted-metal seal or an epoxy seal. Other techniques described below may also apply.

In another possible embodiment, the inventive subject matter provides that the shaft may include a cobalt base alloy with a significant amount of chromium. In another embodiment, the shaft may include about 20 wt.-% Chromium, about 35 wt.-% Nickel, and about 35 wt.-% Cobalt. In another possible embodiment, the shaft may include cobalt base alloy MP35N™. In another embodiment, the shaft may be adapted to deliver a guided device to a region comprising the ureter, bladder, kidney, or gynecological areas of a patient.

In another possible embodiment, the inventive subject matter contemplates an endoscope including a proximal end having an operational interface; a distal end having a working element; and an elongated, semi-rigid, one-piece shaft, between the distal end and the proximal end, comprising a cobalt base alloy. In another embodiment, the endoscope may be adapted for use as a ureteroscope. In another embodiment, the endoscope may be hermetically sealed via at least one melted metal joint joining a portion of the shaft comprising the cobalt base alloy to another portion.

In another example embodiment, the endoscope may further include a housing connected to the shaft via an hermetically sealed joint. In the foregoing embodiment, the housing may be made in whole or part from stainless steel and/or aluminum.

Another embodiment of the inventive subject matter contemplates a ureteroscope having a proximal end that includes an eyepiece and a light guide connector post; a distal end that includes a distal lens; a semi-rigid shaft including two working channels, optical imaging fibers, and light carrier fibers; and the shaft comprising a cobalt base alloy.

Further contemplated embodiments are directed to a medical device including a first portion comprising a super alloy, and a second portion comprising a dissimilar metal joined to the first portion by a hermetic seal, such as may be provided by an arc welding technique. The device may be an endoscope. Additionally, the first portion allows a distal portion of the device to flex relative to a proximal portion.

In another embodiment, the inventive subject matter contemplates a medical device that includes a first portion comprising a cobalt base alloy, and a second portion comprising a non-metal joined to the first portion via an hermetic seal. In this embodiment, the first portion may allow a distal portion of the device to flex relative to a proximal portion; the non-metal may be an optically transmissive material, such as glass or sapphire. The distal portion of such a medical device may further include at least one working element and the proximal portion may include at least one operational interface.

The inventive subject matter also contemplates a method for making a medical device, by providing a first component of the medical device comprising a cobalt base alloy and providing a second component of the medical device comprising a metal dissimilar to the cobalt base alloy, and joining the components by a hermetic seal, such as may be provided, for example, by an arc welding technique. In another method, the arc welding technique may be a Tungsten Inert Gas welding technique, such as a micro Tungsten Inert Gas welding technique for example. Other techniques as described below may also apply.

The inventive subject matter further contemplates a medical device, including a joint of a cobalt base alloy and a dissimilar material effected by a hermetic sealing technique, such as an arc welding technique. In the foregoing embodiment, the arc welding technique may include a Tungsten Inert Gas welding technique; for example, a micro Tungsten Inert Gas welding technique.

The inventive subject matter also contemplates a method of making an endoscope, by providing a first component of the endoscope comprising a cobalt base alloy and providing a second component of the endoscope comprising a metal dissimilar to the cobalt base alloy and joining the components using an arc welding technique. In another method, the arc welding technique may be a Tungsten Inert Gas welding technique, such as a micro Tungsten Inert Gas welding technique for example.

These and other embodiments are described in more detail in the following detailed descriptions and the figures. The foregoing is not intended to be an exhaustive list of embodiments and features of the inventive subject matter. Persons skilled in the art are capable of appreciating other embodiments and features from the following detailed description in conjunction with the drawings.

### BRIEF DESCRIPTION OF THE DRAWINGS

The following figures show various embodiments of inventive subject matter (except where prior art is noted).

FIG. 1a shows an example embodiment of an endoscope, in which the inventive subject matter may be used.

FIG. 1b shows an enlarged detail of the distal end with working elements of the endoscope of FIG. 1a.

FIG. 2 shows a side view of an example embodiment of a medical device, in which the inventive subject matter may be used, having a handle and a working element.

FIGS. 3a-3f show additional details of example embodiments of working elements for use at the end of a shaft.

FIG. 4 shows a side view of another endoscope, in which the inventive subject matter may be used.

FIG. 5a-5b show a detail of the front of the distal end of the embodiment shown in FIG. 4.

FIG. 6 shows a perspective side view of another endoscope, in which the inventive subject matter may be used.

FIG. 7 shows a side view of an embodiment of an elongated shaft assembly according to the inventive subject matter disclosed herein.

FIG. 8 is a cross-section taken along the line A-A in FIG. 7.

FIG. 9 is a cross-section taken along the line B-B in FIG. 7.

FIG. 10a shows a perspective view of the shaft of FIG. 7 with a housing at the proximal end.

FIG. 10b shows a side view of a detail of the shaft and housing shown in FIG. 10a.

FIG. 11 is a front view of the shaft assembly of FIG. 10a.

FIG. 12 shows an enlarged side view of the distal end of the shaft of FIG. 10a.

FIG. 13 is a cross-sectional view, taken along line A-A of FIG. 10b.

FIG. 14 is an enlarged front view of the distal end a welded shaft and forceps tube assembly.

FIG. 15 is a cross-sectional view at the central section of a welded shaft and forceps tube assembly.

FIG. 16 shows a side view of a light carrier welded assembly.

FIG. 17 shows a top view of the light carrier welded assembly of FIG. 16.

FIG. 18 shows a front view at the distal end of the light carrier welded assembly of FIG. 16.

FIG. 19 shows a side view of a semi-rigid autoclaveable ureteroscope.

FIG. 20 shows a cross-sectional view along the line A-A of the distal end of the ureteroscope of FIG. 19.

### DETAILED DESCRIPTION

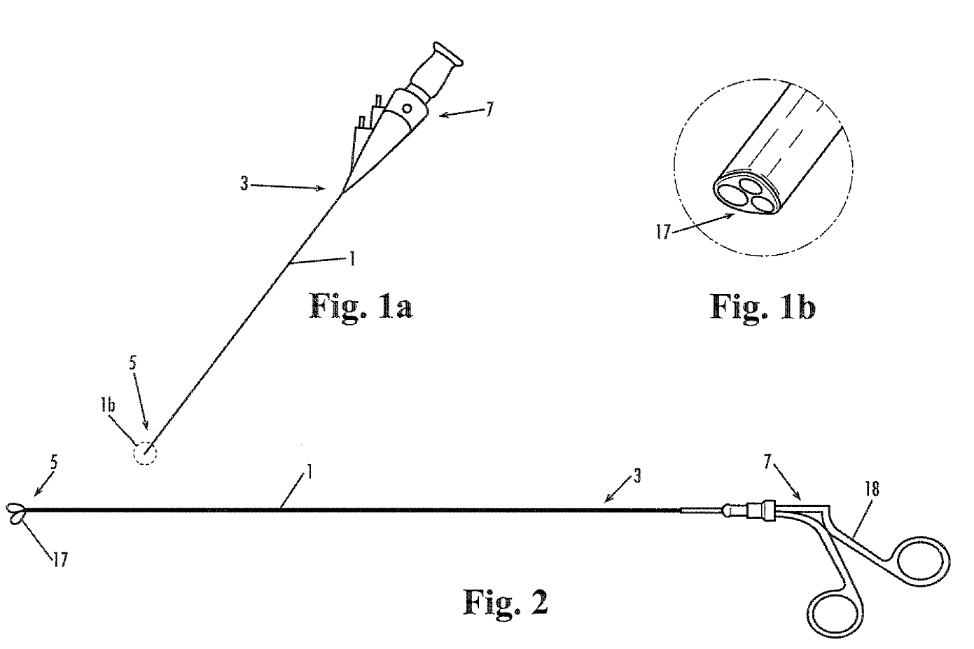

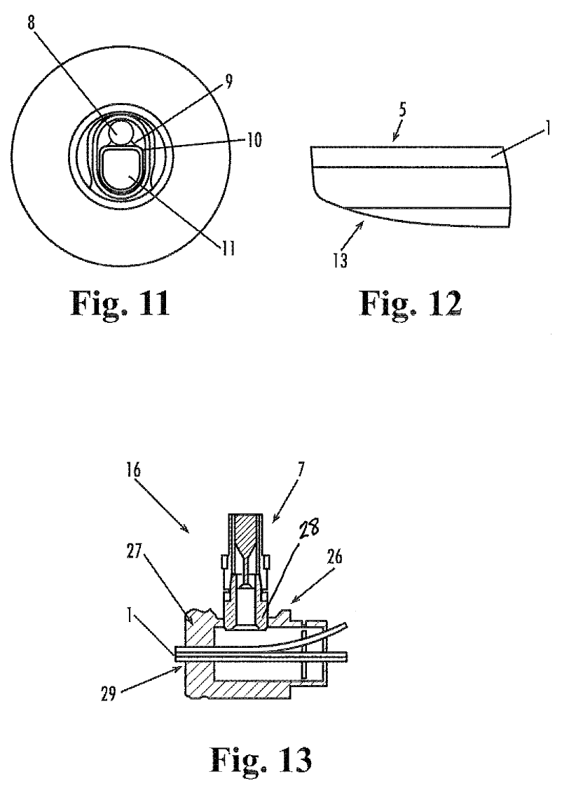

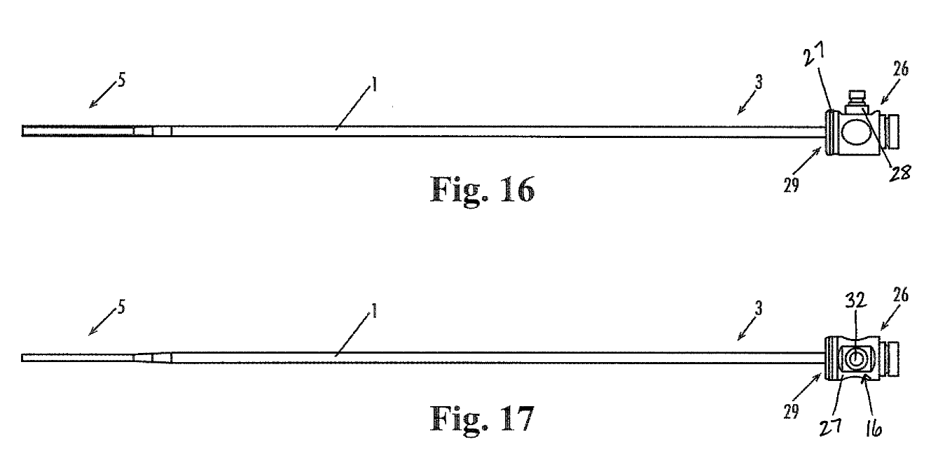

Representative embodiments of the inventive subject matter are shown in FIGS. 1-20, wherein the same or similar features share common reference numerals.

The inventive subject matter primarily relates to novel shaft assemblies for medical or surgical instruments, devices, or tools wherein the shaft in whole or part is formed from a super alloy, which provides deformability and fatigue resistance. Such devices include, but are not limited to, endoscopes, grasping and retrieval devices, and cutting and/or ablation devices (mechanical and electrosurgical), particularly those used in minimally invasive surgery. In certain respects the inventive subject matter is directed to shaft assemblies that include at least a section formed from a semi-rigid material of a super alloy, such as cobalt base alloy. More detail about suitable super alloys is provided below.

A medical device, such as an endoscope for example, may have an elongated shaft with a proximal end and a distal end. The shaft may, in whole or part, be made from a cobalt base alloy, such as described in more detail below. The proximal end may have one or more operational interfaces that provide an operational functionality. For example, the operational interface may be a handle, one or more control buttons, switches, an eyepiece, etc. The operational interface may be an element or mechanism for connecting to a machine or apparatus, such as camera or video equipment camera, vacuum source, fluid source, display devices, computers, robotic arm, etc.

The distal end 5 may have one or more working elements that provide an operational functionality related to the insertion site within a patient or object, as described in more detail below.

The elongated shaft may be solid or may have one or more longitudinal channels, or some combination of hollow, solid, channeled, apertured, compartmented, shapes with the main consideration being that the shaft supports a working functionality at the distal end and an operational functionality at the proximal end. The proximal and distal functionalities need not be disposed at the very ends of the shaft but can be based on any proximal-distal separation along the shaft, or extending away and apart from its ends. The main consideration is whether or not the flexibility of the cobalt base alloy portion of a shaft allows one of the functionalities to deflect relative to the other.

A longitudinal channel in a shaft may provide a conduit or supporting structure for one or more working elements. The working element may be permanently or removably housed in the channel for introduction to a target site. The shaft may also include multiple channels, for example, supporting illumination systems, surgical instruments, suction, irrigation, pull filaments, or for introducing or removing fluids, therapeutic agents, or other substances into a target site. The proximal end of the shaft may further have a control section for controlling the working elements. The shaft may be adapted to deliver a guided device to a region comprising the ureter, bladder, kidney, or gynecological areas of a patient.

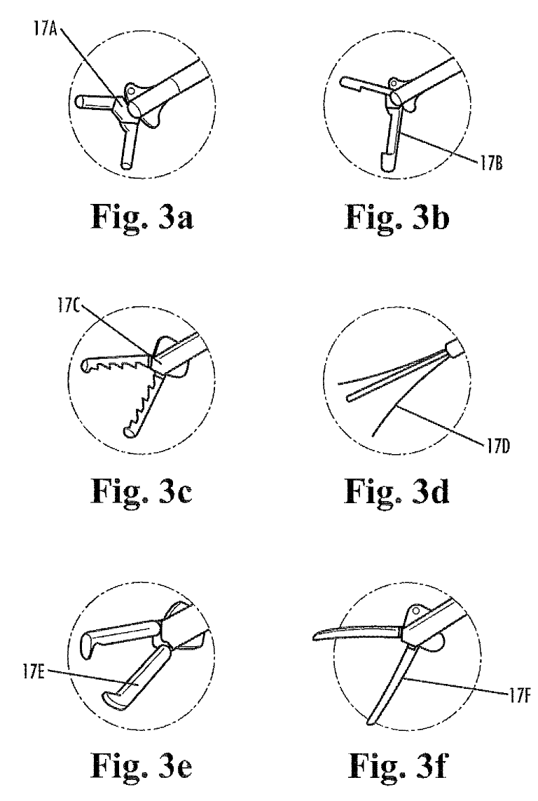

Working elements that may be attached to or integrated with a shaft, or introduced through or over a shaft, include a distal optical port (e.g., a distal window or an objective lens, or other optical components or assemblies) in communication with an optical train in a shaft channel or an imaging system disposed at the distal end of the shaft, which may include an image sensor. Additionally, working elements may include various devices for performing procedures on an object or tissue at a target site in the patient's body. Several examples of working elements are shown in FIG. 1b and FIGS. 3a-f. For instance, a working element may be used for grasping objects such as foreign bodies or stones (calculi) from the patient's body, or it may be used for cutting and retrieving polyps or biopsy samples. FIG. 3a-f shows examples of working elements 17A-17F for a distal end of a shaft for a medical device. Contemplated working elements also include retrieval baskets, biopsy forceps, suction devices, electrosurgical devices, laser devices, and ablation devices. FIG. 1b illustrates the location of certain possible working elements integrated with the shaft of a semi-rigid ureteroscope.

FIG. 2 shows an example of a flexible forceps, which may have a semi-rigid shaft assembly of a cobalt base alloy. The forceps may be used separated from or through an endoscope, and illustrates that the inventive subject matter is not limited to medical devices that are endoscopes. The forceps of FIG. 2 includes an elongated shaft 1 with proximal and distal ends 3 and 5, an operational interface 7, in particular a scissor handle 18, at the proximal end 3, and a working element 17, in particular forceps jaws, at the distal end of the shaft.

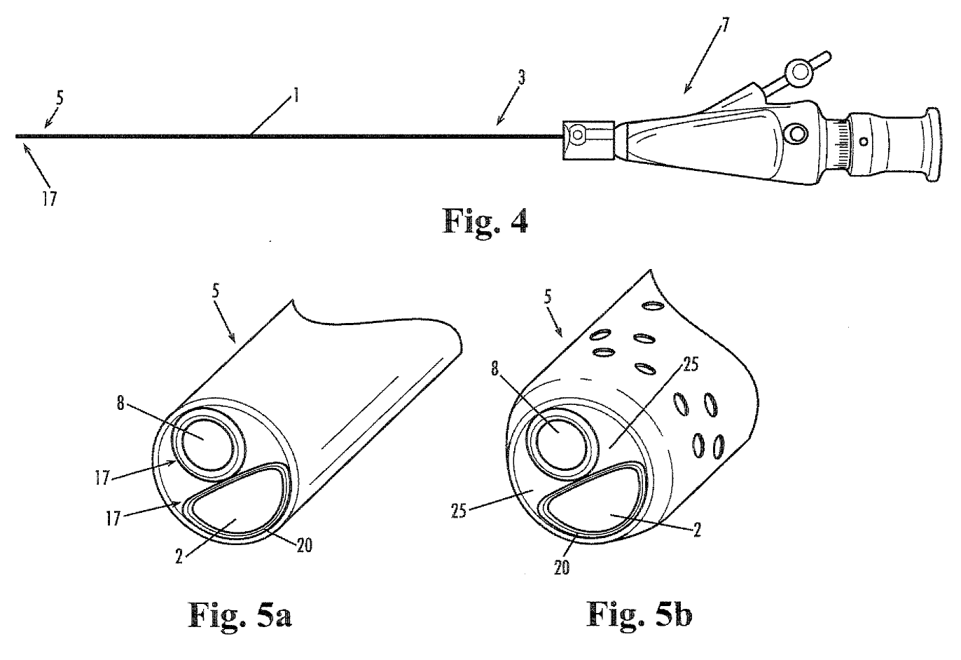

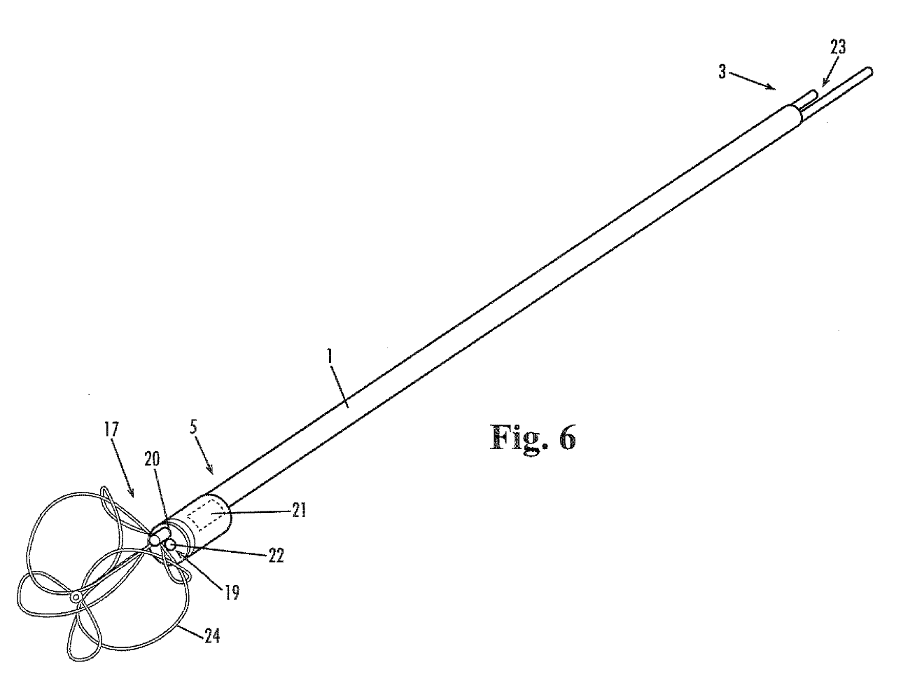

Furthermore, a working element at the distal end of the shaft of an endoscope may be an optical or electronic imaging system and illumination systems. As illustrated in FIGS. 4-5, the imaging system may include an optical view port 8 and an image sensor, such as a CCD, CMOS sensor, or other image acquisition device capable of translating an optical image into an electronic analog or digital signal. Typically the imaging system may further include a distal window; lenses, prisms and other optical elements for transmitting an image; and other imaging components associated with an image sensor, such as supporting electronics and conductors. In some embodiments, the imaging system may be housed in a capsule-like housing disposed at the distal end of the shaft, as illustrated in FIG. 6. In such embodiment, when the physician passes the capsule into the patient, the orifice or incision temporarily dilates to accommodate the diameter of the capsule. After the capsule passes deeper into the patient toward the site of interest, the orifice or incision relaxes to accommodate the smaller diameter of the shaft. Minimizing capsule diameter, shaft diameter, or both limits the amount and duration of the stretching and pressure imposed on patient tissue. Representative imaging system technology that may be used in endoscopes is disclosed in U.S. Pat. No. 6,659,940 and PCT/IL03/00399, which are herein incorporated by reference in their entireties.

The illumination system for an endoscope may be an LED light source or conventional fiber optics connected to an on- or off-board light source. LED illumination systems are disclosed in the aforementioned patent documents.

As the skilled person will be aware, use of CMOS and similar technologies permit the sensor, the transmitting device, the receiving device, the hard wire logic, the driver and the voltage regulator to be integrated into a single semiconductor integrated circuit, and such integration is particularly advantageous in achieving a compact design of endoscope.

An endoscope may further be provided with an image processing system, such as a local or central controller and display unit. The communication between the controller and the display unit may be conducted through a wire connection with an operational interface or a wireless connection. Central control and display unit are typically located on a rack in the operating room. The image processing system typically executes, among other tasks, basic reconstruction of the image including color reconstruction, interface to the user, display of the video and additional data, manual/automatic control over image acquisition parameters, and image reconstructing algorithms for improving resolution and local contrast based on the specific design of the sensor.

FIGS. 4 and 5 show an endoscope having an operational interface 7 at the proximal end. At the distal end, the endoscope has a working elements assembly 17, which is shown in detail in FIG. 5 and includes a fiberoptic lens assembly. The distal end includes two working elements, in particular an imaging system port 8 and a distal port 20. In this example, the port 8 is associated with fiber optics that receive an image through the port. The fiber optics extend to the proximal end of the device where they are coupled to an imaging or view or display system. The working element assembly 17 also includes a working element in the nature of distal port 20 (opening) for a longitudinal channel 2 that runs from a proximal end opening to a distal end opening, to allow insertion of a working tool, such as the forceps of FIG. 2. Advantageously, the shaft of the forceps or other insertable device can be made with a cobalt base alloy so that it may deflect with the deflection of the shaft of the endoscope.

FIG. 6 shows an endoscope with another example of a working element assembly 17, including an imaging system 19, having an optical view port 22 in optical communication with an image sensor 21, and a port 20 at the distal end of a channel for an insertable functional element, such as a retrieval basket device 24. In some embodiments, the proximal end of the shaft may be connected to a handle (not shown) so that one or more pull wires 23 or other filamentous elements are operatively connected between the handle and shaft for controlled deflection of the shaft. In other embodiments, the handle may be omitted so that electrical or electronic filaments terminate in connectors and actuators at the proximal end of the shaft that permit operation of an actuated device.

Shafts used for the instruments and devices as illustrated may have uniform or varying cross-section along its length. For example, it may have a larger cross section in a proximal region and a smaller cross-section more distally, to facilitate distal flexibility for navigating tortuous passages. The construction and arrangement of example semi-rigid shafts is further detailed below.

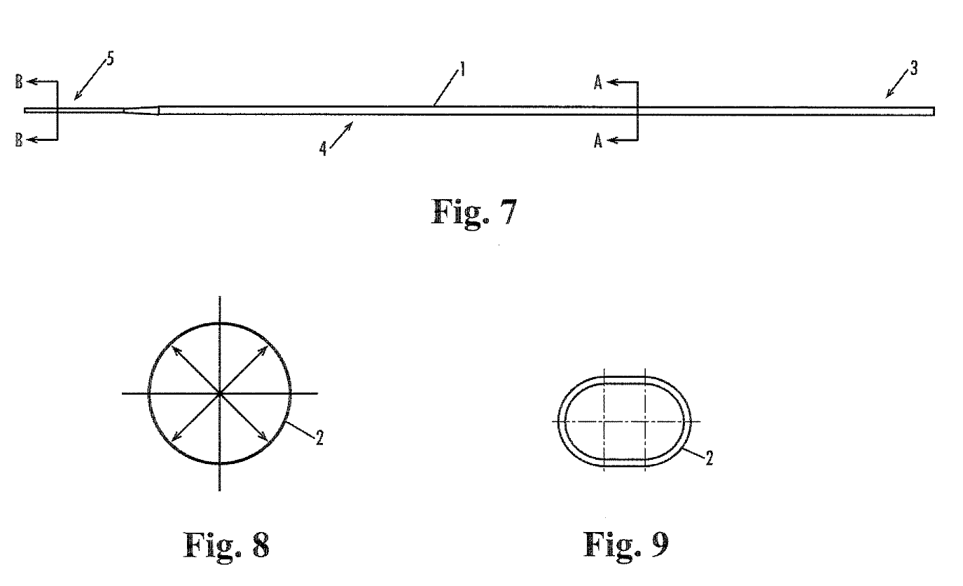

FIG. 7 shows an elongated shaft 1 of a ureteroscope having a longitudinal channel 2. The channel may be defined by a single piece of tubing. The shaft 1 has a proximal end 3, a central section 4, and a distal end 5. In this specific embodiment, the shaft 1 is a thin-walled, tube-like covering made out of one-piece of cobalt alloy tubing. However, the cobalt alloy tubing may also form a segment of a longer shaft structure. The tubing may have an electro-polish finish.

Typical examples of ureteroscopes in which such a shaft 1 may be used include those offered for sale by Gyrus-ACMI Corporation, Southborough, Mass., which are identified as catalog numbers ACMI MR-6A, ACMI MR-6LA, ACMI MRO-742A, and ACMI MRO-733A. The shaft may be adapted in any known or desired manner, for example with two working channels, a proximal eyepiece, optical imaging fibers, light guide connector post, a distal lens, and light carrier fibers.

In one example embodiment, shown in FIGS. 7-9, the channel 2 may have a circular cross-sectional shape in central section 4, with a diameter of about 0.148 inches. The cross-sectional shape of the channel 2 in the distal section 5 may be oblong. For certain minimally invasive procedures, example dimensions are as follows: a width of about 0.139 inches and a height of about 0.095 inches. The shaft 1 includes a transition zone 6, which provides for a tapered transition between the circular central section 4 and the oblong distal end 5. However, the cross-section of the shaft and working channel may have any geometrical shape desired and the length of the shaft may be adapted for a particular use.

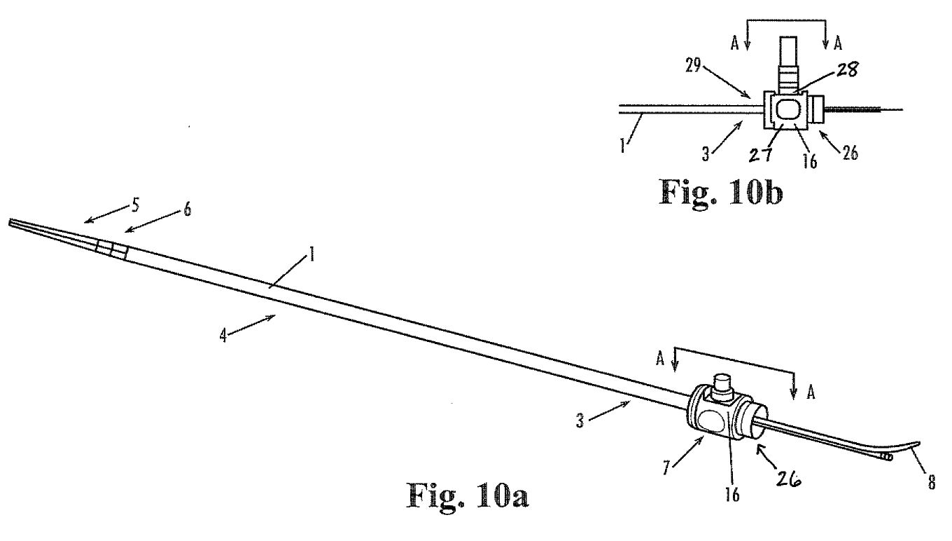

FIGS. 10-13 illustrate another representative embodiment of a ureteroscope according to the inventive subject matter, having a proximal end with an operational interface assembly 7 that includes a light carrier assembly 16 for coupling an illumination system to an off-board light source. The shaft 1 is provided with an image system tube 8, two light fibers 9 and 10, and a channel 11 for an insertable device, such as a forceps or guide wire. FIG. 12 shows a side view of the distal end tip 13 of the shaft 1, which is slightly rounded.

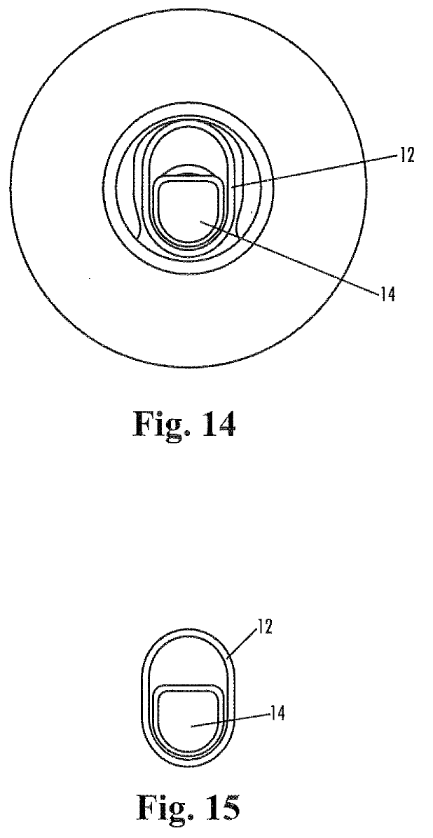

In other example embodiments, as shown in FIGS. 14-15, a ureteroscope may be provided with a shaft 1 having a forceps tube 12 and a working channel 14, allowing one or more guide wires to pass through.

FIGS. 19-20 illustrate a semi-rigid autoclaveable ureteroscope, including a proximal end 3 with an eyepiece 30 and a light guide connector post 32; a distal end 5 including a distal lens 34; and an elongated, semi-rigid, one-piece shaft including two working channels 36 and 38, optical imaging fibers, and light carrier fibers. The elongated shaft may include a cobalt base alloy. The ureteroscope may, for example, have an image system port 8 at the distal end 5, with an optical train 15, including an optically transmissive material 40.

Creating a hermetically sealed endoscope, which withstands the harsh conditions of autoclaving or other sterilization techniques, presents several challenges. In particular, different parts of an endoscope ideally require different material attributes. Some desirable materials that can withstand autoclave conditions may not easily join to other desirable materials, at least not in a hermetic seal. Examples of materials that may need to be joined are metal-to-metal, glass-epoxy-metal, metallized glass-metal, and metal to alloy.

As discussed above, the distal end portion of the endoscope typically includes at least part of an imaging system 19, such as an optical view port 22, for example, an optically transmissive material, such as glass or sapphire, and a distal port 20, for introducing a working tool to a target site. It is desirable to hermetically seal this particular area of the endoscope.

In one particular embodiment, illustrated in FIG. 5b, a hermetic seal may be obtained by using a bonding agent. A suitable bonding agent is an epoxy material as a filler to join components. In this example, the distal end assembly is hermetically sealed by an epoxy filler 25 sealing the area between the distal port 20 and the inner diameter of the shaft, and around image system port 8. The hermetic seal may be achieved by using an epoxy material that resists the sterilization cycles of an autoclave. For example, the epoxy material may be heated and applied to the distal end when fluid. After hardening, the epoxy material may be polished or sandblasted to obtain the desired finish. An example of such an epoxy material is EPO-TEK™353ND provided by Epoxy Technology, Inc. of Billerica, Mass.

Other hermetic joints for the distal end assembly or other assemblies in the device generally may be achieved by soldering, metallizing non-metal materials and soldering and welding, adhesives, electron beam welding, and press fit. As used herein “metal” means an elemental metal or an alloy of elemental metals. One way of joining dissimilar metal materials may be achieved by using solid-state welding techniques. Solid-state welding is a group of techniques that produces coalescence at temperatures below the melting point of the base materials being joined, without the addition of a filler material. It includes friction welding, cold welding, diffusion welding, explosion welding, forge welding, hot pressure welding, roll welding, and ultrasonic welding. It is believed that some or all of these techniques may be used to form a hermetic enclosure for a medical instrument, depending on which dissimilar metals are to be joined. Another option for joining materials is by laser beam welding. This technique joins pieces of metal through the use of a laser. The beam provides a concentrated heat source. Laser beam welding has high power density resulting in small heat-affected zones and high heating and cooling rates. When this technique was applied to joining cobalt base alloy with stainless steel, the resulting bond shows micro-fractures potentially allowing ingress of substances during autoclaving. Therefore, other techniques were required to overcome the problems posed by the conventional technique. The following discussion illustrates the construction of an example inventive medical device based on a cobalt base alloy and a dissimilar metal.

Certain hermetically sealed endoscopes are disclosed in PCT published application number WO2006/066022, which is herein incorporated as reference as if set forth in its entirety for all purposes.

For clarity, the figures depict a generalized embodiment. It is understood, however, that working elements and channels may be added or omitted to adjust the shaft diameter. In other embodiments, channels may not be necessary at all, for example, when working with wireless technology. Moreover, in certain embodiments, the elongated shaft may have a solid core. Some embodiments of the inventive subject matter may contemplate the use of a passive shaft, namely one that has no active provision for directional control. Other embodiments may contemplate the use of a deflecting shaft, that is, one that includes a mechanism for actively bending the distal end of the shaft.

The material used for the shafts or other flexible portions of a medical device of the inventive subject matter is a super alloy. The use of a super alloy enables construction of shafts or other parts that are semi-rigid and fatigue resistant. Generally, “super alloys” are alloys with superior mechanical strength and creep resistance, i.e. resistance to permanent deformation, at high temperatures (above about 700° C.); good surface stability; and corrosion and oxidation resistance. Super alloys typically have a face-centered cubic and austenitic crystal structure. A super alloy's base alloying element is usually nickel, cobalt, or nickel-iron. Examples of super alloys are available from Super Alloys International Limited, Milton Keynes, United Kingdom. (http://www.superalloys.co.uk/products.htm).

One suitable super alloy is a cobalt base alloy. As used herein, a “cobalt base alloy” means an alloy in which cobalt is a substantial component. In this regard “substantial” means that cobalt is present in a percentage greater than about 15% by weight. (All other percentages of elements in this document are also weight percentages.) Particularly suitable cobalt base alloys are those in the category of “super alloys”, which contain in addition to the base cobalt also contain a significant amount of chromium. A significant amount of chromium means at least about 5%. Chromium's role generally is to passivate the alloy, making it more corrosion resistant.

One suitable composition of a cobalt base alloy for use, for example, in a shaft for a ureteroscope is about 35 wt.-% each cobalt and nickel, about 20 wt.-% chromium, and about 10 wt.-% molybdenum, but many other cobalt base alloys are believed suitable. Additional elements, such as carbon, may be included to acquire the desired characteristics of the alloy. The shaft of one example embodiment, as shown in FIGS. 7-20, may be composed of an elongated tube having MP35N™, which is a commercially available super alloy of cobalt and chromium. The MP35N™ tube may have an electro-polish finish. MP35N™ is a trademark of SPS Technologies, Inc. of Jenkintown, Pa. Under the Unified Numbering System for Metals and Alloys, the number for this cobalt base alloy is UNS R30035.

The primary requirement for the cobalt base alloy for use in constructing a semi-rigid part, such as a shaft is that, regardless of composition, the part can be configured to combine a desired modulus of elasticity, level of stiffness, torqueability, and flexibility. In particular, the material used should show biocompatible, balancing elasticity and rigidity, and having reversible deformation abilities. Additionally, the alloy should exhibit high fatigue strength, while avoiding being too brittle. There are a range of commercially available cobalt base alloys which may be selected from.

The cobalt base alloy provides a shaft with adequate column strength, flexibility, and torque resistance to be inserted into a patient's body. The memory capacity of the cobalt base alloy provides for a shaft that returns to its original shape after frequent use. This makes the medical instrument user friendly because the shaft can easily be reconfigured to its original shape (or other desired shape) by the user.

Materials, such as Tinel or Nitinol, for example, are too soft and do not provide the rigidity required to get the shaft to where it needs to go and may not provide sufficient strength to protect on-board components, assemblies or systems.

MP35N™ alloy possesses a unique combination of ultrahigh tensile strength, up to 300 ksi (2068 MPa), good ductility and toughness, and corrosion resistance. This alloy resists corrosion in hydrogen sulphide, salt water and other chloride solutions. Additionally, it features exceptional resistance to stress corrosion cracking at very high strength levels under severe environmental conditions that can crack most conventional alloys. MP35N™ is also highly resistant to other forms of localized attack, such as pitting and crevice corrosion. In seawater environments, this alloy is virtually immune to general, crevice and stress corrosion, regardless of strength level or process condition. The density of MP35N™ is about 8.43 g/cm³. The melting point of MP35N™ is 1440° C. The modulus of elasticity ranges between 201×10³ MPa and 235×10³ MPa.

In addition to cobalt base super alloys described above, it is also expected that many nickel base super alloys, or other super alloys, are suitable for use in the devices contemplated herein, and persons skilled in the art may select such alloys in view of the teachings herein.

In one example embodiment, a medical device, for example an endoscope, may have a first portion including a super alloy, and a second portion including a dissimilar metal joined to the first portion. Typically, in the case of a endoscope, the parts are joined via a hermetic seal, as discussed elsewhere herein. The first portion may allow a distal portion of the device to flex relative to a proximal portion. In another embodiment, a medical device may include a first portion having a cobalt-base alloy, and a second portion having a non-metal joined to the first portion. Here also, the first portion may allow a distal portion of the device to flex relative to a proximal portion. The non-metal may include an optically transmissive material, such as glass or sapphire.

The elongated shaft using a super alloy may be manufactured in any one of a number of ways. In one method of manufacturing the raw material is shaped into a one-piece solid tubular structure. A longitudinal channel may be obtained in the center of the shaft by gun drilling, but could be formed by any suitable means. The tube may be drawn to the desired thickness. The raw MP35N™ alloy and other super alloys are available through known providers, such as SPS Technologies, Inc, and Carpenter Technology Corporation of Wyomissing, Pa. Suppliers of the alloys in preformed tubular structures include manufacturers, such as Medical Metals LLC of Ridgefield, Conn. and Accellent Inc. of Wilmington, Mass.

The MP35N™ alloy may be manufactured by a three-step process. An initial melting process uses Vacuum Induction Melting (VIM) techniques, followed by an Electro Slag Remelt (ESR) to remove some impurities. The process may be followed by Vacuum Arch Remelting (VAR). The triple-melt practice is thought to give best overall performance for this alloy. These cobalt-based alloys develop a highly polished appearance as they are drawn to fine diameters.

In order to measure the durability and resilience of the shaft material, the following tests were conducted on semi-rigid ureteroscopes as illustrated by FIGS. 7-20. The following are test results of attempts to make an acceptable cobalt base shaft. These are intended as guide posts to help those skilled in the art arrive at suitable shaft construction. The test results herein are believed to be accurate and reproducible. However, the test results are intended to supplement the foregoing teachings, and, as is always the case with any experiment, may not have been carried out in a manner that is perfectly in accordance with scientific principles or otherwise beyond question. Accordingly, the following test results should be viewed as prophetic in nature, but the Applicant reserves the right to rely on them as actual experiments should it be necessary to overcome certain kinds of rejections that might arise during the examination of this patent document.

First, a test was conducted on a MR-6A shaft of MP35N™ regarding its shaft loading durability. This particular ureteroscope is autoclavable, and it features improved optical characteristics, and a hermetically sealed outer body. The shaft showed no permanent deflection until an average 4.5-pound force had been applied to the MP35N™ shaft. The test demonstrates the load characteristics of the MP35N™, material “memory” properties, as well as to establish the expected load range to find the MP35N™ shaft material performed well beyond the specified minimum expectation of the current MR-6 stainless steel shaft design.

Second, a cyclic shaft flexure test was conducted on a MR-6LA endoscope. Repeated use cycles subject the endoscope to flexure and methods for cleaning, disinfecting, and sterilizing. Sterilization by placing the endoscope in an autoclave subjects the instrument to high temperature and pressure, which can damage or degrade the endoscope. Endoscopes provided with this super alloy of cobalt base alloy, however, show significantly higher repeat use cycles than similar endoscopes with stainless steel shafts. In particular, a ureteroscope provided with shaft comprising MP35N™ cobalt base alloy allowed over 20,000 cycles of flexure while retaining satisfactory image quality. Similar ureteroscopes, having a stainless shaft, would have failed after a couple of hundred cycles of flexure.

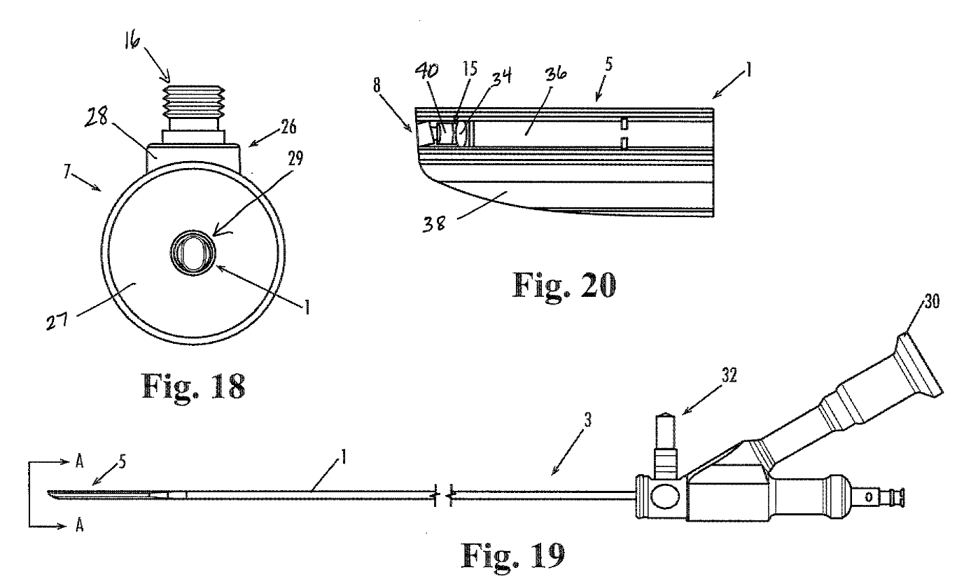

In one example of constructing an endoscope, a first component of an endoscope including a cobalt base alloy is joined to a second component including a dissimilar metal by an arc welding technique and forming a portion of an assembly for an operational interface of one or more working elements. In this example, the assembly includes a housing based on stainless steel and/or aluminum, which is disposed at the proximal end of the cobalt base alloy shaft. FIGS. 13 and 18 show a detail of an operational interface assembly 7. In this particular embodiment, a stainless steel hub or housing portion 26 supports functionalities, such as a light post and a guide wire tube. As shown in FIG. 13, the housing 26 has a first portion 27 and a second portion 28. A weld 29 joins the stainless steel first portion 27 of the housing 26 to the cobalt alloy shaft 1. It is desirable that the weld 29 forms a hermetic seal. Certain hermetically sealed endoscopes are disclosed in PCT published application number WO2006/066022, which is herein incorporated as reference as if set forth in its entirety for all purposes.

Some endoscopes may be hermetically sealed via melted metal joints. A melted metal or coalesced metal seal may be used to connect a housing portion to the shaft. “Melted-metal seal” herein refers to any joint formed by any of various soldering, brazing, welding, or fusion techniques such as laser welding and gold soldering. However, in other embodiments, different materials may need to be joined to create an hermetically sealed endoscope. A laser welding process, such as for example used to weld a stainless steel housing to a stainless steel handle, may not be effective in joining a cobalt base shaft to a stainless steel housing. Due to the high cooling rates, cracking may be a concern when welding high carbon steels. In particular, when trying to join a cobalt base alloy shaft to a stainless steel housing micro-cracking may occur. Therefore, a laser beam welding technique may be effective to hermetically join elements of certain similar material, such as stainless steel. However, this technique may not form an hermetic closure when trying to join dissimilar materials, such as a cobalt base alloy shaft to a stainless steel housing portion.

The inventive subject matter overcomes this problem by a novel method and construction for joining dissimilar metals in a construction for a medical device. In one possible approach according to the inventive subject matter, the novel welding of a cobalt base alloy to a dissimilar metal is achieved using an arc welding process, in particular micro Tungsten Inert Gas (TIG) welding. Arc welding refers to a group of welding processes that use a welding power supply to create an electric arc between an electrode and the base material to melt the metals at the welding point. Both conventional TIG and micro TIG welding processes use tungsten and inert gas to produce an arc. The arc is formed between the non-consumable tungsten electrode and the metal being welded. Gas, usually argon, is fed through the welding torch to shield the molten weld pool and electrode. A filler material matching the base material is usually used. A wide variety of welding wires that provide filler are commercially available, including wires based on cobalt alloy and stainless steel alloy. Micro TIG welding is designed for precision welding of small and thin parts. It differs from conventional TIG welding by the amp range, the size of the filler rod and tungsten. The amp range in conventional TIG welding is about 50 to 250 amps, as compared to the range for micro TIG welding, which is between 5 and 50 amps. Micro TIG welding also uses a filler rod that measures between 0.005 and 0.015 inch in diameter, while TIG welding uses rods that typically measure 0.0625 to 0.125 inch in diameter. Additionally, micro TIG welding uses optics and lighting to aid in applying a precise weld. The micro TIG welding process offers the advantage of joining the metals at a lower amperage and a cooler process temperature. This allows for a more controlled beam with the advantage of avoiding microscopic cracks. Power supplies and control systems for micro TIG welding process are available from, for example Pro Fusion Technologies, Inc. (Idaho). The process is performed using inert shielding gas and controlled weld schedules that yield consistent clean welds. All welds are leak tested to drawing specifications using a Helium Mass Spectrometer to verify their integrity.

It is also believed that electron beam welding is another option to join a cobalt alloy shaft to stainless steel or other dissimilar metal.

An example of a welded assembly is illustrated in FIGS. 10a-b, 13, 16-18, where a first portion 27 of a housing 26 is micro TIG welded to a shaft 1, while other parts of the housing are laser welded together. The weld joints may be tested for hermetic sealing by using a helium leak detector.

Persons skilled in the art will recognize that many modifications and variations are possible in the details, materials, and arrangements of the parts and actions which have been described and illustrated in order to explain the nature of this inventive subject matter and that such modifications and variations do not depart from the spirit and scope of the teachings and claims contained therein. All patent and non-patent literature cited within this application is hereby incorporated by reference as if listed in its entirety herein.

1. A medical device comprising:
a shaft having a proximal end and a distal end,
the proximal end having an operational interface;
the distal end having a working element and
the shaft comprising a super alloy.

2. The medical device of claim 1, wherein the shaft is solid.

3. The medical device of claim 1, wherein the shaft comprises a longitudinal channel.

4. The medical device of claim 1, wherein the operational interface comprises a handle.

5. The medical device of claim 1, further comprising a control section at the proximal end for controlling the working element.

6. The medical device of claim 1, wherein the working element comprises a port in communication with a channel disposed in the shaft.

7. The medical device of claim 1, further comprising an imaging system disposed at the distal end of the shaft.

8. The medical device of claim 7, wherein the operational interface is connected or connectable to an image processing system.

9. The medical device of claim 7, wherein the imaging system comprises an imaging sensor.

10. The medical device of claim 7, wherein the imaging system comprises an optical view port.

11. The medical device of claim 1, further comprising a housing joined to the shaft at a shaft portion comprising a super alloy, and the housing portion joined to the shaft comprising a metal dissimilar from the super alloy.

12. The medical device of claim 11, wherein the shaft and the housing are joined by an arc welding process.

13. The medical device of claim 12, wherein the shaft and the housing are joined by a Tungsten Inert Gas welding process.

14. The medical device of claim 13, wherein the shaft and housing are joined by a micro Tungsten Inert Gas welding process.

15. The medical device of claim 1, wherein the shaft comprises a cobalt base alloy with a significant amount of chromium.

16. The medical device of claim 1, wherein the shaft comprises about 20 wt.-% Chromium, about 35 wt.-% Nickel, and about 35 wt.-% Cobalt.

17. The medical device of claim 1, wherein the shaft comprises cobalt base alloy MP35N™.

18. The medical device of claim 1, wherein the shaft is adapted to deliver a guided device to a region comprising the ureter, bladder, kidney, or gynecological areas of a patient.

19. An endoscope comprising:
a proximal end having an operational interface;
a distal end having a working element; and
an elongated, semi-rigid, one-piece shaft, between the distal end and the proximal end, comprising a cobalt base alloy.

20. The endoscope of claim 19, wherein the endoscope is adapted for use as a ureteroscope.

21. The endoscope of claim 19, wherein the endoscope is hermetically sealed via at least one melted metal joint joining a portion of the shaft comprising the cobalt base alloy to another portion.

22. The endoscope of claim 19, further comprising a housing connected to the shaft via an hermetically sealed joint.

23. The endoscope of claim 22, wherein the housing comprises stainless steel.

24. The endoscope of claim 22, wherein the housing comprises aluminum.

25. The endoscope of claim 22, wherein the housing comprises stainless steel and aluminum.

26. A ureteroscope comprising:
a proximal end including an eyepiece and a light guide connector post;
a distal end including a distal lens;
a semi-rigid shaft including two working channels, optical imaging fibers, and light carrier fibers; and
the shaft comprising a cobalt base alloy.

27. A medical device comprising:
a first portion comprising a super alloy, and
a second portion comprising a dissimilar metal joined to the first portion via an hermetic seal.

28. The medical device of claim 27, wherein the device is an endoscope, and the hermetic seal is provided by an arc welding technique.

29. The medical device of claim 27, wherein the first portion allows a distal portion of the device to flex relative to a proximal portion.

30. A medical device comprising:
a first portion comprising a cobalt base alloy, and
a second portion comprising a non-metal joined to the first portion via an hermetic seal.

31. The medical device of claim 30, further comprising a distal portion to the first portion and a proximal portion to the second portion wherein the first portion allows the distal portion of the device to flex relative to a proximal portion.

32. The medical device of claim 30, wherein the non-metal comprises an optically transmissive material.

33. The medical device of claim 31, wherein the non-metal comprises glass.

34. The medical device of claim 31, wherein the non-metal comprises sapphire.

35. The medical device of claim 31, wherein the distal portion comprises at least one working element and the proximal portion comprises at least one operational interface.

36. A method for making a medical device, comprising:
providing a first component of the medical device comprising a cobalt base alloy;
providing a second component of the medical device comprising a metal dissimilar to the cobalt base alloy;
and joining the components using a technique providing an hermetic seal.

37. The method of claim 36, wherein the technique is an arc welding technique.

38. The method of claim 37, wherein the arc welding technique is a Tungsten Inert Gas welding technique.

39. A medical device, comprising a first component of a medical device and a second component of a medical device, one of the components comprising a cobalt base alloy, and other component comprising a dissimilar material, the components joined via an hermetic seal.

40. The medical device of claim 39, wherein the hermetic seal being provided via a Tungsten Inert Gas welding technique.

41. The medical device of claim 40, wherein the arc welding technique comprises a micro Tungsten Inert Gas welding technique.

42. A method of making an endoscope, comprising: providing a first component of the endoscope comprising a cobalt base alloy and providing a second component of the endoscope comprising a metal dissimilar to the cobalt base alloy and joining the components using an arc welding technique.

43. An endoscope, having a first component comprising a cobalt base alloy and a second component comprising a metal dissimilar to the cobalt base alloy, and a joint between the components effected by an arc welding technique.

44. The endoscope of claim 43, wherein the arc welding technique comprises a Tungsten Inert Gas welding technique.

45. The endoscope of claim 44, wherein the arc welding technique comprises a micro Tungsten Inert Gas welding technique.
